SEQUENCE LISTING (18 sequences)

SEQ ID NO: 1 - DNA, 20 bp, synthetic construct
  source 1..20: mol_type = other DNA
gtgctgttggtcacattgct

SEQ ID NO: 2 - DNA, 20 bp, synthetic construct
  source 1..20: mol_type = other DNA
cagtgccgtcagttcttgtg

SEQ ID NO: 3 - DNA, 21 bp, synthetic construct
  source 1..21: mol_type = other DNA
gcctcacctatgggatcttca

SEQ ID NO: 4 - DNA, 19 bp, synthetic construct
  source 1..19: mol_type = other DNA
tcaaagcctgacgcagatg

SEQ ID NO: 5 - DNA, 21 bp, synthetic construct
  source 1..21: mol_type = other DNA
acacctactttgaagacccat

SEQ ID NO: 6 - DNA, 19 bp, synthetic construct
  source 1..19: mol_type = other DNA
tgacaactttcacctccat

SEQ ID NO: 7 - DNA, 20 bp, synthetic construct
  source 1..20: mol_type = other DNA
tccggacattcaaccatcac

SEQ ID NO: 8 - DNA, 21 bp, synthetic construct
  source 1..21: mol_type = other DNA
tcactgcacatcccagatctc

SEQ ID NO: 9 - DNA, 20 bp, synthetic construct
  source 1..20: mol_type = other DNA
atagcttcaggcgcatttac

SEQ ID NO: 10 - DNA, 20 bp, synthetic construct
  source 1..20: mol_type = other DNA
ttctccatcattctgcatcg

SEQ ID NO: 11 - DNA, 21 bp, synthetic construct
  source 1..21: mol_type = other DNA
gtctcaagtgatgaactgcca

SEQ ID NO: 12 - DNA, 20 bp, synthetic construct
  source 1..20: mol_type = other DNA
ttgagtgctgagtccaggtc

SEQ ID NO: 13 - DNA, 20 bp, synthetic construct
  source 1..20: mol_type = other DNA
ctctggccaagggagatgtt

SEQ ID NO: 14 - DNA, 20 bp, synthetic construct
  source 1..20: mol_type = other DNA
gtcccccaccattgaacttc

SEQ ID NO: 15 - DNA, 21 bp, synthetic construct
  source 1..21: mol_type = other DNA
cagacaaaggagcgcaagaag

SEQ ID NO: 16 - DNA, 21 bp, synthetic construct
  source 1..21: mol_type = other DNA
tgaggcttaagtggtcttgca

SEQ ID NO: 17 - DNA, 20 bp, synthetic construct
  source 1..20: mol_type = other DNA
taccaccatgtacccaggca

SEQ ID NO: 18 - DNA, 24 bp, synthetic construct
  source 1..24: mol_type = other DNA
ctcaggaggagcaatgatcttgat

(19) United States
(12) Patent Application Publication
(10) Pub. No.: US 2026/0021151 A1
(43) Pub. Date: Jan. 22, 2026

(54) COMPOSITION FOR PREVENTING, AMELIORATING OR TREATING LIVER DISEASE, CONTAINING BIOCONVERTED RED GINSENG EXTRACT AS ACTIVE INGREDIENT

(71) Applicant: BTC Corporation, Gyeonggi-do (KR)

(72) Inventors: Tae Young KIM, Seoul (KR); Yong Duk KIM, Gyeonggi-do (KR); Jae Woo LEE, Gyeonggi-do (KR); Yoon Hee KIM, Gyeonggi-do (KR); Yoo KIM, Stillwater, OK (US); Da Yeon LEE, Stillwater, OK (US)

(73) Assignee: Tae Young KIM, Seoul (KR)

(21) Appl. No.: 18/939,323

(22) Filed: Nov. 6, 2024

(30) Foreign Application Priority Data
Jul. 22, 2024 (KR) 10-2024-0096086

(51) Int. Cl. A61K 36/258; A61K 36/062; A61P 1/16; C12N 9/24; C12N 9/42

(52) U.S. Cl. CPC A61K 36/258; A61K 36/062; A61P 1/16; C12N 9/2437; C12N 9/2445; C12N 9/2477; A61K 2236/19; A61K 2236/333; C12Y 302/01004; C12Y 302/01023

(57) ABSTRACT

The present disclosure relates to a composition for preventing, ameliorating or treating a liver disease, which contains a bioconverted red ginseng extract as an active ingredient, specifically a composition for preventing, ameliorating or treating cholestatic liver disease or non-alcoholic fatty liver. The composition of the present disclosure, which contains the bioconverted red ginseng extract as an active ingredient, has the effect of increasing the expression of bile acid biosynthesis and transport-related genes in liver tissue, increasing the relative abundance of Parabacteroides goldsteinii and Ruminococcus faecis strains in the gut, inhibiting the expression of the ApoE gene, and inhibiting the accumulation of lipid droplets in liver tissue. Thus, the composition of the present disclosure can be usefully used in the prevention, amelioration or treatment of liver disease, specifically cholestatic liver disease or non-alcoholic fatty liver.

### CROSS-REFERENCE TO RELATED APPLICATION

This application claims priority to Korean Patent Application No. 10-2024-0096086 filed on Jul. 22, 2024, and all the benefits accruing therefrom under 35 U.S.C. § 119, the contents of which in its entirety are herein incorporated by reference.

### SEQUENCE LISTING

The instant application contains a Sequence Listing which has been submitted electronically in a computer readable Sequence Listing XML format and is hereby incorporated by reference in its entirety. Said computer readable Sequence Listing in XML format was created on Jan. 14, 2025, is named G1035-29401_SequenceListing.xml and is 16,996 bytes in size.

### BACKGROUND

### 1. Field

The present disclosure relates to a composition for preventing, ameliorating or treating a liver disease, which contains a bioconverted red ginseng extract as an active ingredient, specifically a composition for preventing, ameliorating or treating cholestatic liver disease or non-alcoholic fatty liver.

### 2. Description of the Related Art

As the largest organ in the body, the liver is a very important organ responsible for various metabolism, detoxification, breakdown, synthesis and secretion processes. Since the liver has a function of regulating energy metabolism, all nutrients absorbed from food are metabolized into substances capable of producing energy in the liver and supplied or stored throughout the body. The liver is also responsible for the synthesis, storage and distribution of about 2,000 enzymes, albumin, serum proteins of coagulation factors, bile acids, fats such as phospholipids or cholesterol, etc.

Bile, which is secreted by the liver, contains bile acids, phospholipids, bilirubin, GSH, electrolytes, etc. It is not only a route for the excretion of toxic substances and lipolysis products but also participates in the digestion and absorption of fats in the gut, thereby performing a variety of physiological functions, excretes endogenous wastes such as bilirubin and various toxic substances such as drugs, increases normal fat absorption in the gut, and plays a pivotal role in maintaining the equilibrium of cholesterol metabolism (Ballatori N et al., Am. J. Physiol., 263, pp. G617-G624, 1992).

Bile acid metabolites are synthesized or produced under various stressful conditions such as fasting or obesity, alcohol intake, inflammatory diseases and diabetes, and are known to be involved in the regulation of various metabolic diseases (Li T et al., Pharmacol Rev, 2014; 66:948-983).

Cholestatic liver disease (cholestasis) refers to biochemical, physiological or clinical changes caused by impaired circulation of bile. Specifically, cholestatic liver injury is a condition caused by the accumulation of bile acids and lipids. The liver regulates key lipid metabolism processes, including fatty acid synthesis, mitochondrial β-oxidation, and phospholipid transport. Impaired bile secretion, caused by biliary obstruction or liver damage, disrupts cholesterol and phospholipid metabolism, which can lead to non-alcoholic fatty liver.

Primary biliary cirrhosis (PBC) or primary cholestatic cirrhosis, which is a representative cholestatic liver disease, is an autoimmune disease of the liver characterized by the slow, progressive destruction of the small biliary tracts of the liver, along with the intralobular ducts, in the early stage of the disease. If these ducts are damaged, bile can accumulate in the liver (cholestasis), damaging the tissue over time, which can lead to scarring, fibrosis and cirrhosis.

There is currently no known cure for cholestatic liver disease other than liver transplantation. Treatment using drugs is limited to reducing the symptoms of the disease and preventing complications.

Therefore, there is a need for the development of a natural product-derived substance that can prevent, ameliorate or treat cholestatic liver disease or non-alcoholic fatty liver.

### REFERENCES OF RELATED ART

### Patent Documents

(Patent document 001) KR 10-2022-0031311 A.

### SUMMARY

The present disclosure is directed to providing a food composition for preventing or ameliorating cholestatic liver disease or non-alcoholic fatty liver, which contains a bioconverted red ginseng extract as an active ingredient.

The present disclosure is also directed to providing a feed composition for preventing or ameliorating cholestatic liver disease or non-alcoholic fatty liver, which contains a bioconverted red ginseng extract as an active ingredient.

The present disclosure is also directed to providing a pharmaceutical composition for preventing or treating cholestatic liver disease or non-alcoholic fatty liver, which contains a bioconverted red ginseng extract as an active ingredient.

The present disclosure provides a food composition for preventing or ameliorating cholestatic liver disease or non-alcoholic fatty liver, which contains a bioconverted red ginseng extract as an active ingredient, wherein the bioconverted red ginseng extract is one enzymatically reacted by adding an enzyme mixture of cellulase, β-glucosidase and hemicellulase to a red ginseng extract.

According to an exemplary embodiment of the present disclosure, the red ginseng extract may be one extracted with water, a C₁₋₄ lower alcohol, or a mixed solvent thereof.

According to an exemplary embodiment of the present disclosure, the enzyme mixture may be derived from Aspergillus niger.

According to an exemplary embodiment of the present disclosure, the enzyme mixture may be sumizyme AC.

According to an exemplary embodiment of the present disclosure, the bioconverted red ginseng extract may increase the expression level of one or more bile acid biosynthesis-related proteins selected from CYP27A1, CYP46A1 and FXRα, or the genes encoding them.

According to an exemplary embodiment of the present disclosure, the bioconverted red ginseng extract may increase the expression level of one or more bile acid transport-related proteins selected from CATP1A4, OSTα, SOD2, and SOD3, or the genes encoding them.

According to an exemplary embodiment of the present disclosure, the bioconverted red ginseng extract may increase the relative abundance of one or more strains selected from Parabacteroides goldsteinii and Ruminococcus faecis in the gut.

According to an exemplary embodiment of the present disclosure, the bioconverted red ginseng extract may suppress Firmicutes and increase Bacteroidetes among the gut microbiota.

The present disclosure also provides a feed composition for preventing or ameliorating cholestatic liver disease or non-alcoholic fatty liver, which contains a bioconverted red ginseng extract as an active ingredient, wherein the bioconverted red ginseng extract is one enzymatically reacted by adding an enzyme mixture of cellulase, β-glucosidase and hemicellulase to a red ginseng extract.

The present disclosure also provides a pharmaceutical composition for preventing or treating cholestatic liver disease or non-alcoholic fatty liver, which contains a bioconverted red ginseng extract as an active ingredient, wherein the bioconverted red ginseng extract is one enzymatically reacted by adding an enzyme mixture of cellulase, β-glucosidase and hemicellulase to a red ginseng extract.

The composition of the present disclosure, which contains the bioconverted red ginseng extract as an active ingredient, has the effect of increasing the expression of bile acid biosynthesis and transport-related genes in liver tissue, increasing the relative abundance of Parabacteroides goldsteinii and Ruminococcus faecis strains in the gut, inhibiting the expression of the ApoE gene, and inhibiting the accumulation of lipid droplets in liver tissue. Thus, the composition of the present disclosure may be usefully used in the prevention, amelioration or treatment of cholestatic liver disease or non-alcoholic fatty liver.

### BRIEF DESCRIPTION OF THE DRAWINGS

FIG. 1 is a schematic diagram of an experimental method using an animal model of aged mice to identify the efficacy of a bioconverted red ginseng extract (BRG) according to an exemplary embodiment of the present disclosure.

FIG. 2 shows a result of administering a bioconverted red ginseng extract (BRG) according to an exemplary embodiment of the present disclosure to an animal model of aged mice and then analyzing the change of genes in liver tissue.

FIG. 3 shows a result of administering a bioconverted red ginseng extract (BRG) according to an exemplary embodiment of the present disclosure to an animal model of aged mice and then analyzing the change in the expression of genes involved in bile acid biosynthesis in liver tissue.

FIG. 4 shows a result of administering a bioconverted red ginseng extract (BRG) according to an exemplary embodiment of the present disclosure to an animal model of aged mice and then analyzing the change in the expression of genes involved in bile acid transport in liver tissue.

FIG. 5 shows a result of administering a bioconverted red ginseng extract (BRG) according to an exemplary embodiment of the present disclosure to an animal model of aged mice and then analyzing the change of the gut microbiota composition in feces.

FIG. 6 shows a result of administering a bioconverted red ginseng extract (BRG) according to an exemplary embodiment of the present disclosure to an animal model of aged mice and then analyzing the change in the composition of Verrucomicrobiota in feces.

FIG. 7 shows a result of administering a bioconverted red ginseng extract (BRG) according to an exemplary embodiment of the present disclosure to an animal model of aged mice and then analyzing the change in the composition of Akkermansia in feces.

FIGS. 8A to 8D show a result of administering a bioconverted red ginseng extract (BRG) according to an exemplary embodiment of the present disclosure to an animal model of aged mice at different concentrations and then analyzing the change in the composition of the gut microbiota in feces, wherein FIG. 8A shows a result based on the phylum, FIG. 8B based on the family, FIG. 8C based on the genus, and FIG. 8D based on the amplicon sequence variants (ASVs).

FIG. 9 shows a result of administering a bioconverted red ginseng extract (BRG) according to an exemplary embodiment of the present disclosure to an animal model of aged mice at different concentrations and then analyzing the alpha diversity (observed ASVs, Peilou's evenness index and Shannon index) of the gut microbiota in feces.

FIGS. 10A and 10B show a result of administering a bioconverted red ginseng extract (BRG) according to an exemplary embodiment of the present disclosure to an animal model of aged mice at different concentrations and then analyzing the beta diversity of the gut microbiota in feces (FIG. 10A: graphs, FIG. 10B: table).

FIG. 11 shows a result of administering a bioconverted red ginseng extract (BRG) according to an exemplary embodiment of the present disclosure to an animal model of aged mice along with a high-fat/high-calorie diet and then analyzing the change in the expression of the ApoE gene in liver tissue.

### DETAILED DESCRIPTION

The inventors of the present disclosure have completed the present disclosure after discovering that an extract of red ginseng bioconverted using specific enzymes maintains and improves bile homeostasis and inhibits fat accumulation in liver tissue.

The present disclosure will now be described in detail.

An aspect of the present disclosure relates to a food composition for preventing or ameliorating cholestatic liver disease or non-alcoholic fatty liver, which contains a bioconverted red ginseng extract as an active ingredient, wherein the bioconverted red ginseng extract is one enzymatically reacted by adding an enzyme mixture of cellulase, β-glucosidase and hemicellulase to a red ginseng extract.

The bioconverted red ginseng extract, also known as a bioconverted enzyme extract, is a product obtained by reaction of a red ginseng extract as a substrate with the enzyme mixture described above.

According to an exemplary embodiment of the present disclosure, the red ginseng may be one processed by steaming undried 4-6 year-old fresh ginseng for a long time with steam or other methods and then drying to a moisture content of 14% or lower. The red ginseng may be red ginseng root and/or red tail ginseng root, specifically a mixture of red ginseng root and red tail ginseng root at a weight ratio of 50 to 90:10 to 50, more specifically 70 to 80:20 to 30.

According to an exemplary embodiment of the present disclosure, the red ginseng extract may be prepared by mixing red ginseng and an extraction solvent at a weight ratio of 1:1 to 20, specifically 1:12 to 18, more specifically 1:14 to 16, conducting extraction once or multiple times while stirring at 20 to 100° C., specifically 70 to 90° C., for 1 to 15 hours, more specifically 9 to 13 hours, and then concentrating the same under reduced pressure. If the weight ratio of the red ginseng and the extraction solvent is outside the above range, the amount of the extracted active ingredients of red ginseng may decrease.

According to an exemplary embodiment of the present disclosure, the red ginseng extract may be extracted with water, a C₁₋₄ lower alcohol, or a mixed solvent thereof. The lower alcohol may be 30 to 80% (v/v), specifically 40 to 60% (v/v), methanol, ethanol, butanol or propanol.

30 to 80%, specifically 40 to 80%, ethanol is preferred as the extraction solvent for extraction of the active ingredients of red ginseng, although not being limited thereto.

Specifically, the red ginseng extract is concentrated under reduced pressure to 50 to 70 brix.

According to an exemplary embodiment of the present disclosure, the enzyme mixture may be derived from Aspergillus niger, and may specifically be obtained from a culture of Aspergillus niger.

Furthermore, the enzyme mixture may be specifically sumizyme AC (ShinNippon, enzyme mixture of cellulase, β-glucosidase and hemicellulase, produced by Aspergillus niger). Since the sumizyme AC is effective in converting saponins to saponin metabolites and the bioconverted red ginseng extract treated with the sumizyme AC is effective in maintaining or improving bile acid homeostasis and inhibiting fat accumulation in liver tissue, the bioconverted red ginseng extract may be usefully used for preventing, ameliorating or treating cholestatic liver disease or non-alcoholic fatty liver.

Specifically, the addition amount of the enzyme mixture may be 0.01 to 70 parts by weight, specifically 0.1 to 60 parts by weight, more specifically 0.5 to 50 parts by weight, more specifically 1 to 40 parts by weight, more specifically 5 to 30 parts by weight, more specifically 10 to 25 parts by weight, more specifically 15 to 25 parts by weight, based on 100 parts by weight of the red ginseng extract of 50 to 70 brix.

Further, the enzymatic reaction may be carried out at 15 to 70° C., specifically 30 to 67° C., even more specifically 40 to 65° C., even more specifically 45 to 55° C., pH 2 to 7, specifically pH 2.5 to 6, even more specifically pH 3.5 to 5, for a period of from 5 to 120 hours, specifically from 1 to 90 hours, even more specifically from 5 to 60 hours, even more specifically from 20 to 48 hours, even more specifically from 20 to 30 hours.

In carrying out the above enzymatic reaction, if the reaction temperature and/or pH is outside the above range, the activity of the enzyme may be inhibited.

Further, in carrying out the enzymatic reaction, if the reaction time is outside the above range, the conversion rate to saponin metabolites may be significantly lower.

The enzyme type, addition amount, reaction temperature and reaction time described above are optimal conditions in terms of the efficacy of the bioconverted red ginseng extract of the present disclosure for maintaining or improving bile homeostasis and inhibiting fat accumulation in liver tissue. If any of the above conditions is not met, the expected effect of maintaining or improving bile homeostasis and inhibiting fat accumulation in liver tissue may not be achieved.

The bioconverted red ginseng extract is heated at 85 to 95° C. for 20 to 80 minutes to inactivate the enzyme.

In the bioconverted red ginseng extract of the present disclosure prepared in this manner, the sum of the contents of Rb1, Rg1 and Rg3 is at least 7.0 mg/g, specifically 7.0 to 15.0 mg/g, more specifically 9.0 to 13.0 mg/g, the content of F2 is at least 2.0 mg/g, specifically 2.0 to 5.0 mg/g, the content of compound K is at least 1.0 mg/g, specifically 1.0 to 5.0 mg/g, the content of ginsenoside Rk1 is at least 5.0 mg/g, specifically 5.0 to 8.0 mg/g, and the content of ginsenoside Rg5 is at least 5.0 mg/g, specifically 5.0 to 8.0 mg/g.

The bioconverted red ginseng extract of the present disclosure is broadly understood to include a processed product of the bioconverted red ginseng extract, such as a powder obtained by drying the bioconverted red ginseng extract.

According to an exemplary embodiment of the present disclosure, the bioconverted red ginseng extract may increase the expression level of one or more bile acid biosynthesis-related proteins selected from CYP27A1, CYP46A1 and FXRα, or the genes encoding them.

Further, the bioconverted red ginseng extract may increase the expression level of one or more bile acid transport-related proteins selected from CATP1A4, OSTα, SOD2 and SOD3, or the genes encoding them.

Furthermore, the bioconverted red ginseng extract may suppress Firmicutes and increase Bacteroidetes among the gut microbiota.

In particular, the bioconverted red ginseng extract may increase the relative abundance of one or more strains selected from Parabacteroides goldsteinii and Ruminococcus faecis in the gut. For reference, Ruminococcus faecis has been shown to inhibit liver damage caused by liver fibrosis, and Parabacteroides goldsteinii has been shown to improve liver inflammation, produce secondary bile acid (7-ketolithocholic acid), maintain bile homeostasis in the gut, and improve barrier function.

Furthermore, the bioconverted red ginseng extract may increase the species diversity of the gut microbiota.

Furthermore, the bioconverted red ginseng extract may inhibit the expression of the ApoE (apolipoprotein E) gene in liver tissue, significantly inhibit the production of lipid droplets in liver tissue, and significantly reduce blood AST and ALT levels.

As used herein, “as an active ingredient” or “containing as an active ingredient” means containing an amount sufficient to achieve the efficacy or activity of the bioconverted red ginseng extract of the present disclosure, and more specifically, containing an amount sufficient to prevent, ameliorate or treat cholestatic liver disease or non-alcoholic fatty liver. In an exemplary embodiment, the daily dosage of the bioconverted red ginseng extract may be from 100 to 2000 mg/kg, specifically from 200 to 1500 mg/kg, more specifically from 250 to 1000 mg/kg. Since the bioconverted red ginseng extract is a natural product and has no adverse effect on the human body even when administered in excessive amounts, the upper and lower limits of the content of the bioconverted red ginseng extract contained in the composition of the present disclosure can be selected appropriately by those skilled in the art.

As used in the present disclosure, the term “prevention” means any action of suppressing or delaying cholestatic liver disease or non-alcoholic fatty liver.

As used herein, the term “improvement” means at least decreasing a parameter associated with the condition being treated, such as the severity of symptoms, by administering a composition containing the bioconverted red ginseng extract of the present disclosure.

The food composition according to the present disclosure can be formulated as a powder, a granule, a tablet, a capsule, etc. using a sitologically adequate and physiologically acceptable adjuvant for use as a functional food. The adjuvant may include an excipient, a disintegrant, a sweetener, a binder, a coating agent, an extender, a lubricant, a glidant, a flavoring agent, etc.

Furthermore, the food composition according to the present disclosure may be, for example, a beverage, an alcoholic beverage, confectionery, a diet bar, a dairy product, meat, chocolate, pizza, ramen, other noodles, a chewing gum, ice cream, etc.

Another aspect of the present disclosure relates to a health functional food for preventing or ameliorating cholestatic liver disease or non-alcoholic fatty liver, which contains the bioconverted red ginseng extract described above as an active ingredient.

Since the “bioconverted red ginseng extract”, “cholestatic liver disease” and “non-alcoholic fatty liver” were described above, description thereof will be omitted to avoid excessive redundancy.

The health functional food refers to a food product made by adding the bioconverted red ginseng extract to food materials such as beverages, teas, spices, chewing gum, confectionery, etc. or preparing the same into a capsule, a powder, a suspension, etc., which exhibits specific health effects when consumed, but unlike ordinary drugs, has no side effect that may occur when taking drugs for a long time. The health functional food of the present disclosure obtained in this way is very useful because it can be consumed on a daily basis. The amount of the bioconverted red ginseng extract added to the health functional food may vary depending on the type of the health functional food. The addition amount may be within a range that does not impair the original flavor of the food. The addition amount is generally in a range of 0.01 to 50 wt %, specifically 0.1 to 20 wt %, of the target food product. Furthermore, in the case of a health functional food in the form of a powder, a granule, a tablet or a capsule, the addition amount is typically in a range of 0.1 to 100 wt %, specifically 0.5 to 80 wt %. In an exemplary embodiment, the health functional food of the present disclosure may be in the form of a pill, a tablet, a capsule or a beverage.

Another aspect of the present disclosure relates to a food composition for maintaining or improving bile acid homeostasis, which contains a bioconverted red ginseng extract as an active ingredient, wherein the bioconverted red ginseng extract is one enzymatically reacted by adding an enzyme mixture of cellulase, β-glucosidase and hemicellulase to a red ginseng extract.

Since the “bioconverted red ginseng extract” and “food composition” of the present disclosure were described above, description thereof will be omitted to avoid excessive redundancy.

The bioconverted red ginseng extract of the present disclosure is effective in maintaining or improving bile acid homeostasis by increasing the biosynthesis and transport of bile acids whose biosynthesis and circulation were reduced due to aging.

Specifically, the bioconverted red ginseng extract of the present disclosure may increase the expression level of one or more bile acid biosynthesis-related proteins selected from CYP27A1, CYP46A1 and FXRα, or genes encoding them, and may increase the expression level of one or more bile acid transport-related proteins selected from CATP1A4, OSTα, SOD2 and SOD3, or genes encoding them.

Another aspect of the present disclosure relates to a feed additive composition for preventing or ameliorating cholestatic liver disease or non-alcoholic fatty liver, which contains a bioconverted red ginseng extract as an active ingredient, wherein the bioconverted red ginseng extract is one enzymatically reacted by adding an enzyme mixture of cellulase, β-glucosidase and hemicellulase to a red ginseng extract.

Since the “bioconverted red ginseng extract”, “cholestatic liver disease” and “non-alcoholic fatty liver” were described above, description thereof will be omitted to avoid excessive redundancy.

As used in the present disclosure, the term “feed additive” includes substances added to feed for the purpose of various effects, such as supplementing nutrients and preventing weight loss, improving the digestibility of fibers in feed, improving milk quality, preventing reproductive disorders and improving conception rate, preventing heat stress in summer, etc. The feed additive composition of the present disclosure corresponds to the supplementary feed according to the Control of Livestock and Fish Feed Act, and may further contain mineral preparations such as sodium bicarbonate, bentonite, magnesium oxide, compound minerals, trace minerals such as zinc, copper, cobalt, selenium, etc., vitamin preparations such as carotene, vitamins A, D and E, nicotinic acid, vitamin B complex, etc., amino acid preparations such as methionine, lysine, etc., fatty acid preparations such as fatty acid calcium salts, etc., live bacteria (lactobacilli), yeast cultures, fungal ferments, yeast preparations, and the like.

The present disclosure also relates to a feed composition containing the feed additive composition.

As used in the present disclosure, the term “feed” refers to any natural or artificial diet, meal, or component of the meal, intended for or suitable for eating, consumption and digestion by animals. Various types of feeds known in the art can be prepared by adding the bioconverted red ginseng extract according to the present disclosure. Specifically, the feed may include concentrate feed, roughage and/or specialty feed, although not being limited thereto.

The concentrated feed includes seed berries, which include seeds including wheat, oats, corn, etc., brans including rice bran, wheat bran, barley bran, etc. obtained as byproducts of grain refining, seedcakes obtained as byproducts after extracting oil from soybean, rape, sesame, linseed, coconut, etc., dregs such as residual starchy matter which is the main component of starch residues from sweet potato, potato, etc., animal feeds such as fish meal, fish dregs, fish soluble, which is a concentrate of fresh liquid obtained from fish, meat meal, blood meal, feather meal, skim milk, dried whey obtained by drying whey, which is the residue obtained after preparation of cheese from milk and casein from skim milk, etc., yeast, chlorella and algae, although not being limited thereto.

The roughage includes fresh green roughage such as wild grass, grass, soilage, etc., root vegetables such as forage turnip, forage beet, rutabaga, which is a variety of turnip, etc., silage, which is stored feed obtained through lactic acid fermentation of green grass, soilage, crops, etc. in a silo, hay obtained by cutting and drying wild grass or grass, straw from crops for stockbreeding, leaves of legumes, etc., although not being limited thereto. The specialty feed includes mineral feeds such as oyster shell, rock salt, etc., urea feeds such as urea or its derivatives such as diureidoisobutane, etc., feed additives which are substances added in trace amounts to mixed feeds to supplement ingredients that may be lacking in natural feeds or to increase the storability of the feeds, and dietary supplements, although not being limited thereto.

The feed compositions for preventing or ameliorating cholestatic liver disease or non-alcoholic fatty liver according to the present disclosure can be prepared by adding the bioconverted red ginseng extract of the present disclosure in appropriate effective concentration ranges according to various feed preparation methods known in the art.

As used herein, the term “containing as an active ingredient” means containing an amount sufficient to achieve the efficacy or activity of the bioconverted red ginseng extract. The daily administration dosage of the bioconverted red ginseng extract of the present disclosure may be from 100 to 2000 mg/kg, specifically from 200 to 1500 mg/kg, more specifically from 250 to 1000 mg/kg.

The feed composition according to the present disclosure can be used for any individual without limitation for preventing or ameliorating cholestatic liver disease or non-alcoholic fatty liver. For example, it can be used for any individual such as a non-human animal, bird, fish, etc., including cow, horse, pig, goat, sheep, dog, cat, rabbit, etc. Specifically, it can be used for a companion animal such as dog, cat, etc.

Another aspect of the present disclosure relates to a pharmaceutical composition for preventing or treating cholestatic liver disease or non-alcoholic fatty liver, which contains a bioconverted red ginseng extract as an active ingredient, wherein the bioconverted red ginseng extract is one enzymatically reacted by adding an enzyme mixture of cellulase, β-glucosidase and hemicellulase to a red ginseng extract.

Since the “bioconverted red ginseng extract”, “cholestatic liver disease” and “non-alcoholic fatty liver” were described above, description thereof will be omitted to avoid excessive redundancy.

The pharmaceutical composition of the present disclosure may be formulated using a pharmaceutically adequate and physiologically acceptable adjuvant in addition to the active ingredient. The adjuvant may include an excipient, a disintegrant, a sweetener, a binder, a coating agent, an extender, a lubricant, a glidant, a flavoring agent, etc.

The pharmaceutical composition may be specifically formulated as a pharmaceutical composition containing one or more pharmaceutically acceptable carrier in addition to the active ingredient described above for administration.

The pharmaceutical composition may be in the form of a granule, a powder, a tablet, a coated tablet, a capsule, a suppository, a liquid, a syrup, a juice, a suspension, an emulsion, a medicinal dropper, an injectable liquid, etc. For example, for formulation into a tablet or a capsule, the active ingredient may be combined with an oral, nontoxic, pharmaceutically acceptable inert carrier such as ethanol, glycerol, water, etc. Furthermore, if desired or necessary, a suitable binder, lubricant, disintegrant and colorant may also be included as a mixture. The binder includes natural sugars such as starch, gelatin, glucose, or beta-lactose, corn sweetener, natural and synthetic gums such as acacia, tragacanth or sodium oleate, sodium stearate, magnesium stearate, sodium benzoate, sodium acetate, sodium chloride, etc., although not being limited thereto. The disintegrant includes starch, methyl cellulose, agar, bentonite, xanthan gum, etc., although not being limited thereto.

A composition formulated as a liquid solution may contain, as a pharmaceutical acceptable carrier which is sterile and biocompatible, saline, sterile water, Ringer's solution, buffered saline, albumin injection solution, dextrose solution, maltodextrin solution, glycerol, ethanol, and mixtures of one or more of these ingredients. Other common additives such as an antioxidant, a buffer, a bacteriostatic agent, etc. may be added, if necessary. In addition, the composition may also be formulated into an injectable formulation such as an aqueous solution, a suspension, an emulsion, etc., a pill, a capsule, a granule or a tablet by further adding a diluent, a dispersant, a surfactant, a binder and a lubricant.

Furthermore, the composition may be formulated depending on the relevant disease or the ingredients using suitable methods in the art as disclosed in Remington's Pharmaceutical Science (Mack Publishing Company, Easton PA).

The pharmaceutical composition of the present disclosure can be administered orally or parenterally. The parenteral administration can be made by intravenous injection, subcutaneous injection, intramuscular injection, intraperitoneal injection, transdermal administration, etc. Specifically, the composition may be administered by oral administration.

The suitable administration dosage of the pharmaceutical composition of the present disclosure varies depending on factors such as the method of formulation, mode of administration, the age, body weight, sex, pathological condition and diet of a patient, administration time, administration route, excretion rate, and response sensitivity. An ordinarily skilled physician can readily determine and prescribe a dosage effective for the desired treatment or prevention. According to a specific exemplary embodiment of the present disclosure, the daily dosage of the pharmaceutical composition of the present disclosure may be 0.001 to 10 g/kg.

Furthermore, the pharmaceutical composition of the present disclosure may be prepared into a unit dose form by formulation using a pharmaceutically acceptable carrier and/or excipient, or may be prepared by introducing them into a multi-dose container. The formulation may be in the form of a solution, suspension or emulsion in an oil or an aqueous medium, or in the form of an extract, a powder, a granule, a tablet or a capsule, and may further contain a dispersant or a stabilizer.

The present disclosure will now be described in detail by way of examples, but the present disclosure is not limited by the following examples.

### EXAMPLE

### Example: Preparation of Bioconverted Red Ginseng Extract (BRG)

### (1) Preparation of Red Ginseng Extract Concentrate

Red ginseng powder was prepared by grinding 6-year-old red ginseng consisting of red ginseng root and red tail ginseng root at a weight ratio of weight ratio 70:30, which was purchased from Geumsan Ginseng Nonghyup. A red ginseng extract was prepared by adding 15 weight equivalents of 50% (v/v) ethanol to the red ginseng powder and conducting extraction at 80° C. for 11 hours. The red ginseng extract was concentrated under reduced pressure at 60° C. to prepare a red ginseng extract concentrate of 60 brix.

### (2) Enzymatic Treatment

The red ginseng extract concentrate obtained in the step (1) was diluted to 5 wt % by adding purified water. After adding sumizyme AC (ShinNippon, enzyme mixture of cellulase, β-glucosidase and hemicellulase, produced by Aspergillus niger, 20% (w/w) based on the red ginseng extract concentrate obtained in the step (1)), the red ginseng extract (5 wt %) was enzymatically treated at 50° C. and pH 4 for 24 hours. The enzymatically treated red ginseng extract was then heated at 90° C. for 60 minutes to inactivate enzymes.

### (3) Filtration, Concentration and Drying

The enzymatically treated red ginseng extract was filtered, concentrated under reduced pressure, and then vacuum-dried at 60 to 90° C. to prepare a bioconverted red ginseng extract.

### TEST EXAMPLES

In the following test examples, all experiments were performed in triplicate, and the results are expressed as mean±standard deviation. Statistical analysis was performed using the SPSS software (ver. 20.0, SPSS Inc., Chicago, IL, USA). The difference between the mean values was tested by one-way ANOVA. p<0.05 was considered significant. In the respective data, *, ** and *** indicate significant differences at p<0.05, p<0.01 and p<0.001, respectively, compared to a control group or a normal group (Student's t-test), and #, ##and ###indicate significant differences at p<0.05, p<0.01 and p<0.001, respectively, compared to a negative control group (Student's t-test).

### Animal Model of Mice With Cholestatic Liver Disease

DDC (3,5-diethoxycarbonyl-1,4-dihydrocollidine) inhibits the activity of ferrochelatase, which inserts Fe into protoporphyrin IX to generate heme, leading to the accumulation of protoporphyrin in the liver, and the accumulation and crystallization of the hydrophobic protoporphyrin in the biliary tract, which can be discharged out of the liver via bile only, induce inflammation and fibrosis around the biliary tract. Since cholestasis, inflammation and fibrosis in liver tissue are induced by the mechanism described above, the DDC diet-induced animal model is one of the most used cholestatic liver disease animal models of rodents.

The DDC-induced animal model of mice with cholestatic liver disease was prepared using 8-week-old C57BL/6 male mice (Samtako, Korea) according to the method of Elisa Pose et al. Briefly, cholestatic liver disease was induced by feeding a standard rodent diet supplemented with 0.1% (w/w) DDC for 7 or 14 days. The mice were subjected to a 12-hour light/dark cycle and given free access to water. The standard rodent diet included intermediate wheatgrass, wheat, corn, corn gluten powder and soybean oil (14% of protein).

### Test Example 1: Histological Analysis of Cholestatic Liver Disease

Long-term supply of DDC induces the obstruction of the biliary duct by creating intraductal porphyrin plugss and, thereby, damaging the biliary epithelium. A toxic accumulation of bile in the biliary duct leads to cholangiocyte activation and ductular reaction proliferation, and is positive for Sirius Red staining and Masson's trichrome staining.

The tissue staining of the cholestatic liver disease animal model was measured by H&E staining (basic tissue staining), Sirius Red staining (collagen), and Masson's trichrome staining (collagen, cytoplasm, muscle fibers, etc.).

After the mice were fed a diet containing 0.1% DDC for 14 days, tissue staining was performed for inflammation, fibrosis, and adhesion molecules around the hepatic portal vein.

To investigate the effect of the bioconverted red ginseng extract (BRG) on cholestatic liver disease, the mice were fed a diet with 0 (DDC-vehicle), 150 mg/kg (DDC-BRG low) or 300 mg/kg (DDC-BRG high) for 3 days prior to DDC treatment (a total of 17 days) and, after 3 days, were fed a diet with DDC (a total of 14 days). After 14 days of the DDC administration, the mice were sacrificed and the liver was harvested to include the portal vein (PV) and biliary ducts of the mice, and the surrounding lesions were quantitatively measured by H&E. The relative percentage of the lesion area compared to the control group (DDC-vehicle) is shown in Table 1 below.

TABLE 1

| | Areal ratio of lesions |
|---|---|
| | (% of control group) |
| Normal group | — |
| Control group (DDC-vehicle) | 100 |
| Group administered with 150 mg/kg of | 47.8** |
| bioconverted red ginseng extract of Example | |
| Group administered with 300 mg/kg of | 26.4*** |
| bioconverted red ginseng extract of Example | |

As shown in Table 1, the DDC-treated control group (DDC-vehicle) showed significantly increased lesions in the hepatic portal vein and biliary tracts of mice as compared to the normal group. In contrast, all the test groups treated with the bioconverted red ginseng extract according to Example of the present disclosure showed significant reduction in the lesions around the hepatic portal vein and biliary tracts as compared to the control group. In particular, the reduction in lesions was significant in the group treated with 300 mg/kg of the bioconverted red ginseng extract according to Example of the present disclosure as compared to the group treated with 150 mg/kg, indicating that the bioconverted red ginseng extract according to Example of the present disclosure reduces lesions in the DDC-treated hepatic portal vein and biliary tracts in a concentration-dependent manner.

From the above results, it was specifically confirmed that the administration of the bioconverted red ginseng extract of the present disclosure has a preventive effect, such as reduction of lesions in the hepatic portal vein and biliary tracts, in cholestatic liver disease.

### Animal Model of Aged Mice

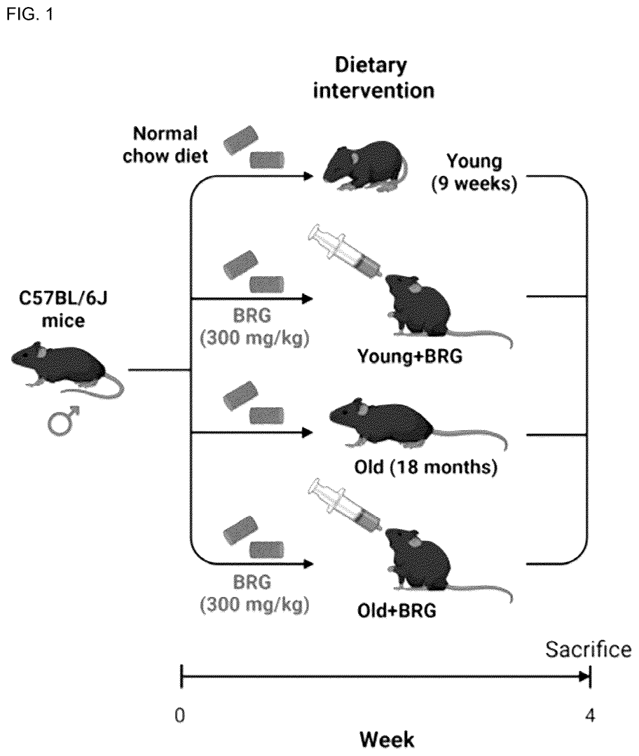

Thirty male C57BL/6 mice aged 2 months (young) and 18-19 months (old) were selected and orally administered with the bioconverted red ginseng extract (BRG) according to Example dissolved in 0.9% physiological saline to a concentration of 300 mg/kg once daily for 4 weeks (Approval No: IACUC-21-34) (FIG. 1). The care of laboratory animals and all experiments were conducted in accordance with the guidelines of the Association for Assessment and Accreditation of Laboratory Animal Care (AAALAC) and were reviewed and approved by the Institutional Animal Care and Use Committee (IACUC) of Oklahoma State University.

### Four Test Groups

Young control group (YC): 0.9% physiological saline was administered to mice (n=10)

Young BRG administration group (young ginseng; YG): 300 mg/kg BRG was administered to young mice (n=10)

Old control group (OC): 0.9% physiological saline was administered to aged mice (n=10)

Old BRG administration group (old ginseng; OG): 300 mg/kg BRG was treated to aged mice (n=10)

### Test Example 2: Analysis of Change of Genes in Liver Tissue

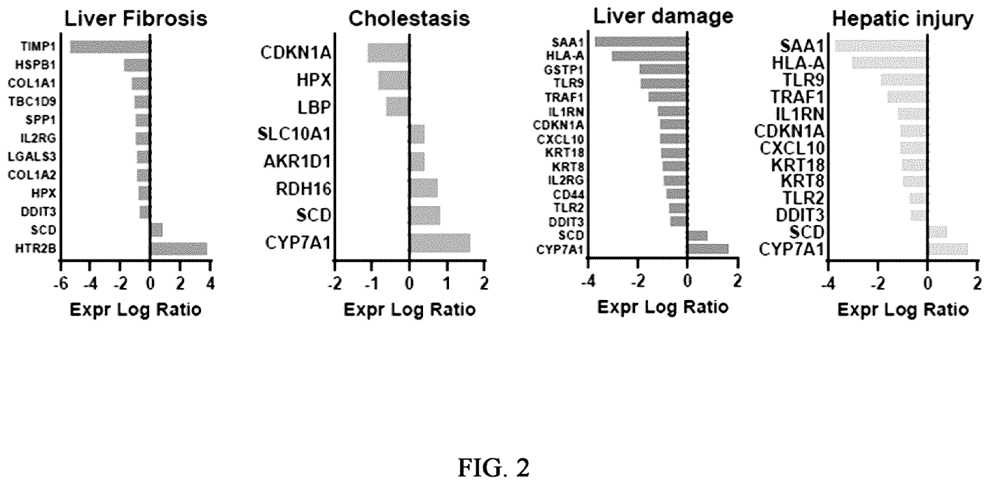

After completion of the 4 weeks of administration, the mice of the old control group (OC) and the old BRG administration group (OG) were sacrificed and total RNA in liver tissue was extracted using the a Qiagen RNeasy mini kit (Hilden, Germany). From the extracted total RNA, significant differentially expressed genes (DEGs) were screened with the Illumina NovaSeq 6000 platform using integrative transcriptome analysis (mRNA sequencing) techniques. The screened DEGs were subjected to gene ontology (GO) analysis, Kyoto Encyclopedia of Genes and Genomes (KEGG) pathway analysis, and ingenuity pathway analysis (IPA) to predict the signaling pathways and subgenes/proteins associated with their transcripts (FIG. 2).

Referring to FIG. 2, it can be seen that the BRG of the present disclosure inhibits TIMP1, HSPB1, COL1A1, TBC1D9, SPP1, IL2RG, etc., which are involved in liver fibrosis in aged mice, inhibits CDKN1A, HPX and LBP, which are involved in cholestatic liver disease, and increases the expression of CYP7A1, SCD, RDH16, etc. Furthermore, it can be seen that the BRG of the present disclosure inhibits SAA1, HLA-A, GSTP1, TLR9 and TRAF1, which are involved in liver damage in aged mice, and increases the expression of CYP7A1, SCD, etc.

### Test Example 3: Analysis of Change in mRNAs Involved in Bile Acid Homeostasis and Improvement of Liver Function

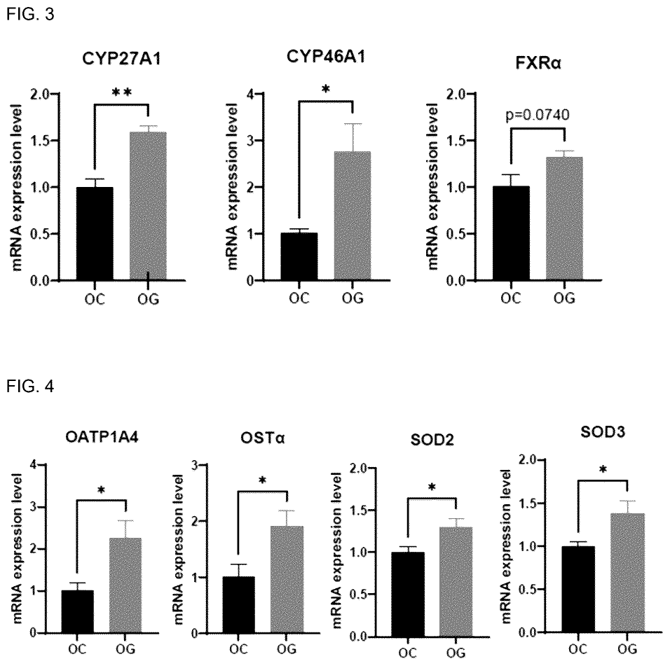

After completion of the 4 weeks of administration, the mice were sacrificed and the expression level of mRNAs associated with bile acid homeostasis in liver tissue was measured. Specifically, liver tissue was homogenized by adding to a TRIzol reagent (Invitrogen, Waltham, MA, USA). The homogenate was mixed with chloroform and centrifuged at 4° C. for 15 minutes at 12,000×g to separate the aqueous layer. The separated aqueous layer was mixed with isopropanol and centrifuged under the same condition. The isolated RNAs were washed with 75% ethanol, dissolved in RNase-free water, and then synthesized into cDNAs using an iScript™ cDNA synthesis kit (Bio-Rad Laboratories, Inc., Hercules, CA, USA). The synthesized cDNAs were subjected to real-time reverse transcription polymerase chain reaction (real-time RT-PCR) to measure the expression level of genes involved in bile acid synthesis (Table 2). The relative gene expression level was calculated as a comparative threshold cycle (Ct) value for β-actin using a CFX Opus 384 real-time PCR system. The result is shown in FIGS. 3 and 4.

TABLE 2

| Target | | Sequence (5′-3′) |
|---|---|---|
| mApoE | Forward | GTGCTGTTGGTCACATTGCT |
| | Reverse | CAGTGCCGTCAGTTCTTGTG |
| mCyp27a1 | Forward | GCCTCACCTATGGGATCTTCA |
| | Reverse | TCAAAGCCTGACGCAGATG |
| mCyp46a1 | Forward | ACACCTACTTTGAAGACCCAT |
| | Reverse | TGACAACTTTCACCTCCAT |
| mFXRα | Forward | TCCGGACATTCAACCATCAC |
| | Reverse | TCACTGCACATCCCAGATCTC |
| mOatp1a4 | Forward | ATAGCTTCAGGCGCATTTAC |
| | Reverse | TTCTCCATCATTCTGCATCG |
| mOsta | Forward | GTCTCAAGTGATGAACTGCCA |
| | Reverse | TTGAGTGCTGAGTCCAGGTC |
| mSOD2 | Forward | CTCTGGCCAAGGGAGATGTT |
| | Reverse | GTCCCCCACCATTGAACTTC |
| mSOD3 | Forward | CAGACAAAGGAGCGCAAGAAG |
| | Reverse | TGAGGCTTAAGTGGTCTTGCA |
| mβ-actin | Forward | TACCACCATGTACCCAGGCA |
| | Reverse | CTCAGGAGGAGCAATGATCTTGAT |

Referring to FIG. 3, it can be seen that the old BRG administration group (OG) shows significantly increased expression of genes involved in bile acid biosynthesis, CYP27A1, CYP46A1 and FXRα, as compared to the old control group (OC).

Furthermore, referring to FIG. 4, it can be seen that the expression of genes involved in bile acid transport, OATP1A4, OSTα, SOD2 and SOD3, was significantly increased in the old BRG administration group (OG) as compared to the old control group (OC).

From the above results, it can be inferred that the administration of the bioconverted red ginseng extract of the present disclosure has the effect of maintaining bile acid homeostasis, and thereby preventing cholestatic liver disease.

### Test Example 4: Microbiome Analysis

### 4-1: Analysis of Change of Microbiome Due to BRG Administration in Aged Mice

For microbiome analysis, feces were collected from the mice at the beginning and end of the BRG administration and genomic DNA was extracted using a QIAamp fast DNA stool mini kit (Qiagen). The extracted genomic DNA was analyzed for 16S rRNA (V3-V4) sequence using next-generation sequencing (NGS) method.

The sequence population of microorganisms obtained by the NGS was analyzed using the QIIME 2 software and expressed as amplicon sequence variant (ASV) values. The relative richness of species was expressed in terms of alpha diversity and beta diversity, which are indicators of the diversity of biological species.

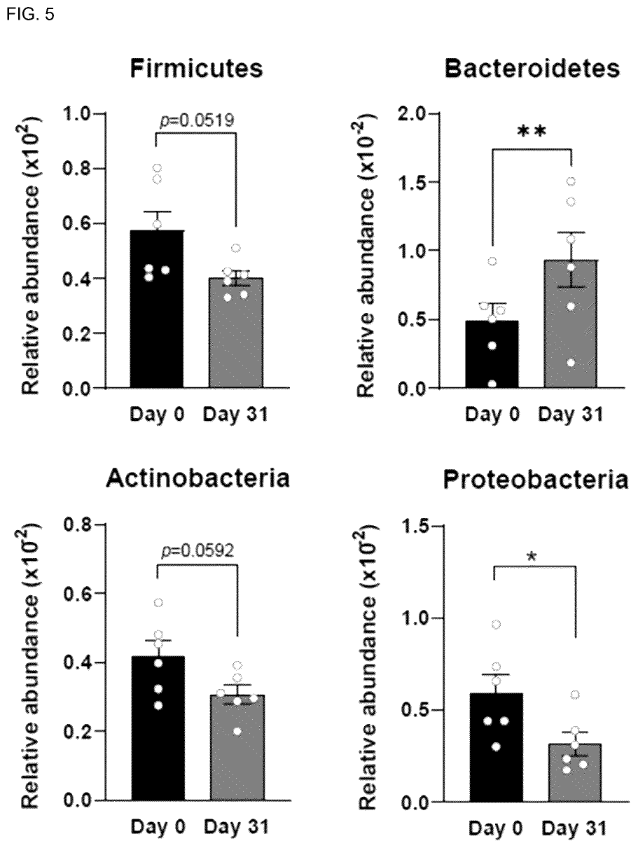

FIG. 5 shows the result of fecal microbiome analysis at the beginning (day 0) and end (day 31) of the BRG administration for the old BRG administration group (OG).

Referring to FIG. 5, it can be seen that the BRG of the present disclosure suppresses Firmicutes, Actinobacteria and Proteobacteria and significantly increases Bacteroidetes among the gut microbiota in aged mice.

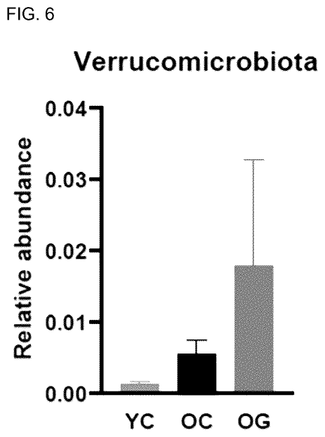

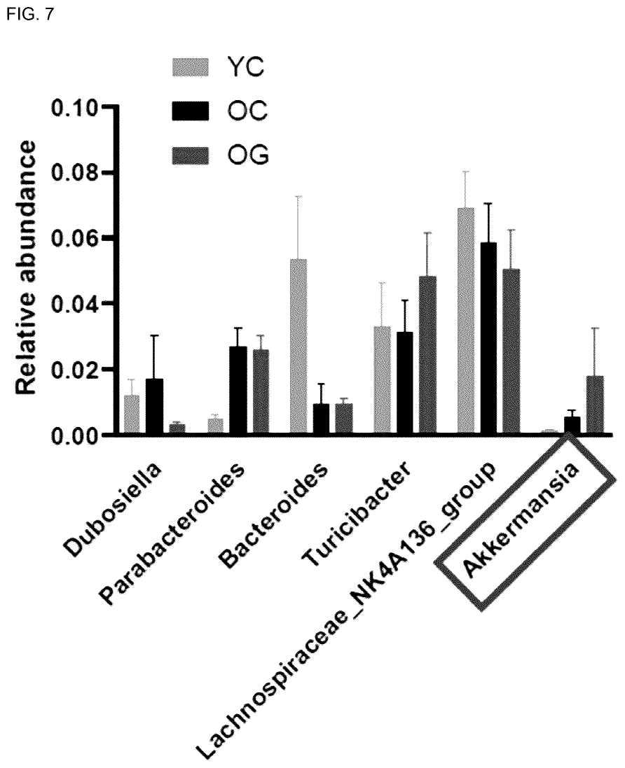

Furthermore, referring to FIGS. 6 and 7, it can be seen that the BRG of the present disclosure significantly increases Verrucomicrobiota and Akkermansia among the gut microbiota in aged mice.

### 4-2: Analysis of Change of Microbiome in Aged Mice Depending on BRG Concentration

Forty male C57BL/6 mice aged 18-19 months (old) were selected and the bioconverted red ginseng extract (BRG) according to Example was administered ad libitum at concentrations of 150 mg/kg and 300 mg/kg for 14 weeks. As a control group, a red ginseng hot water extract (RG) was administered ad libitum at a concentration of 300 mg/kg for the same period.

### Four Test Groups

Control group: 0.9% physiological saline was administered to aged mice (n=10) BRG high-concentration administration group (BRGH): 300 mg/kg BRG was administered to aged mice (n=10)

BRG low-concentration administration group (BRGL): 150 mg/kg BRG was administered to aged mice (n=10)

RG administration group (RG): 300 mg/kg RG was administered to aged mice (n=10)

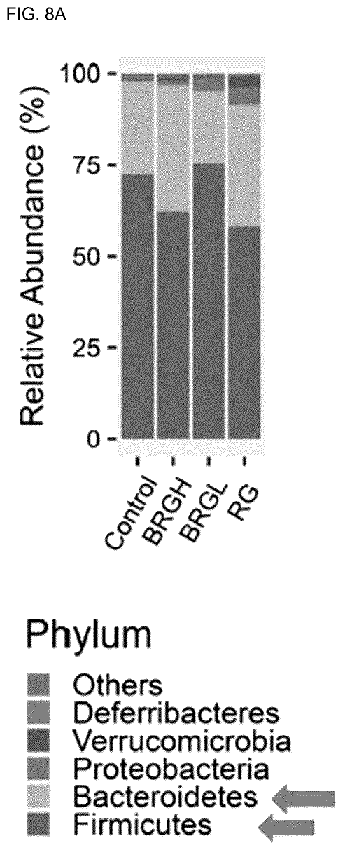

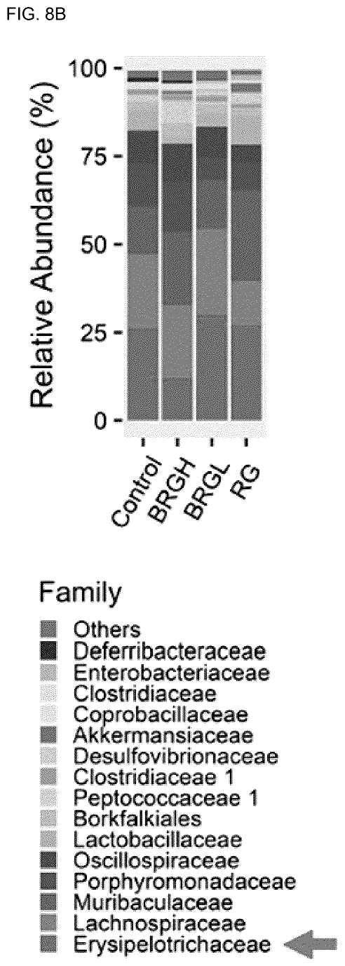

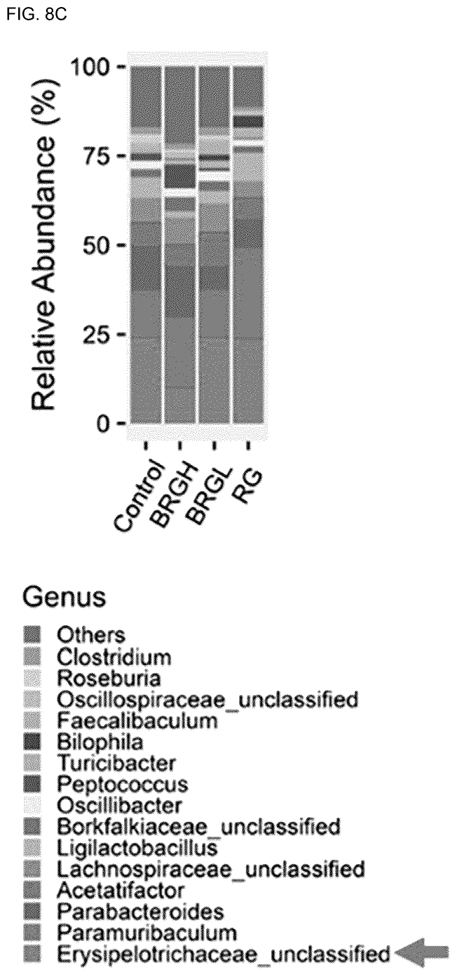

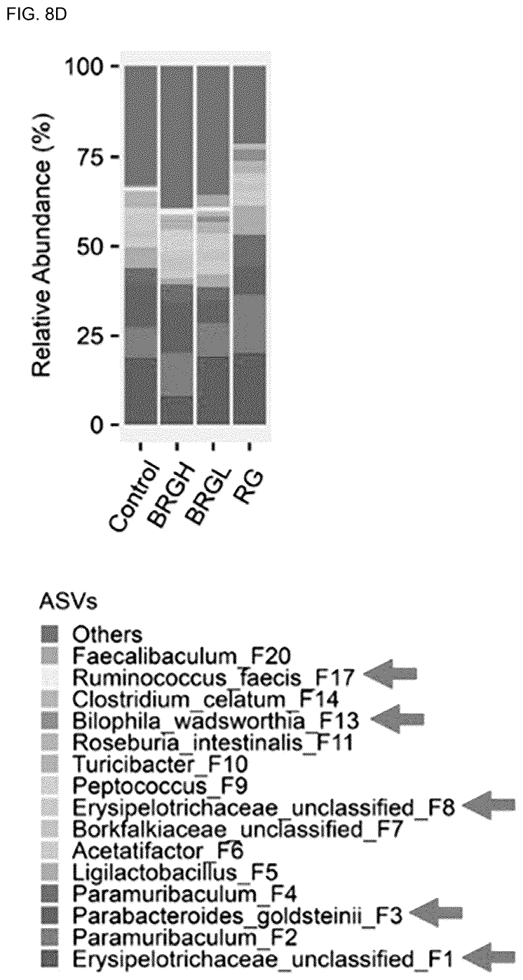

Referring to FIG. 8A, it can be specifically seen that the administration of BRG of the present disclosure at high concentration (BRGH) significantly suppresses Firmicutes and significantly increases Bacteroidetes among the gut microbiota in aged mice.

Furthermore, it can be seen from FIGS. 8B and 8C that the administration of the BRG of the present disclosure at high concentration (BRGH) significantly inhibits Erysipelotrichaceae among the gut microbiota in aged mice.

Furthermore, referring to FIG. 8D, it can be seen that the administration of the BRG of the present disclosure at high concentration (BRGH) significantly increases the relative abundance of Ruminococcus faecis and Parabacteroides goldsteinii among the gut microbiota in aged mice. Specifically, it was confirmed that Ruminococcus faecis increased by about 70% as compared to the control group, and Parabacteroides goldsteinii increased by about 15% compared to the control group.

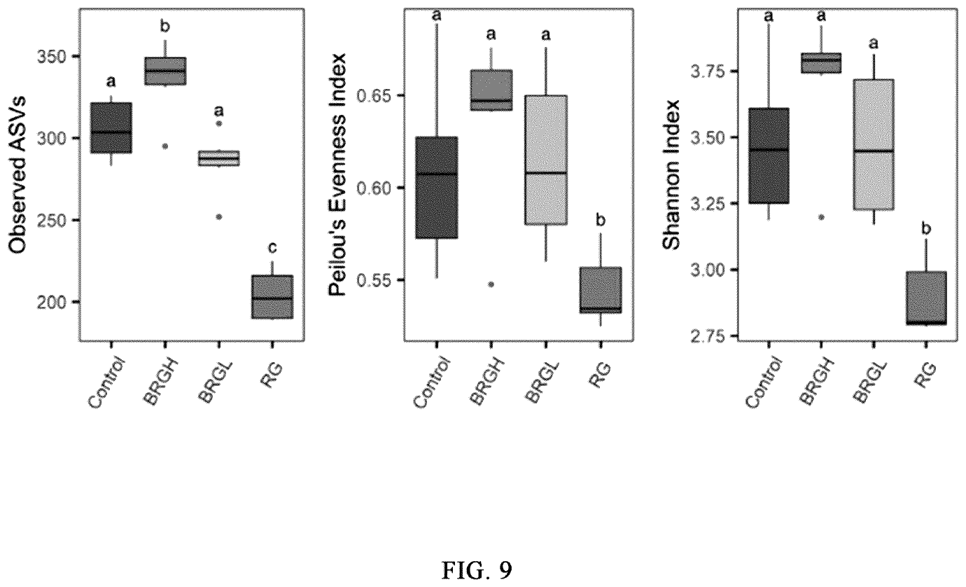

Furthermore, referring to FIG. 9, it can be seen that the administration of BRG at high concentration (BRGH) significantly increased species diversity (alpha diversity), including the number (species richness) and relative abundance (species evenness) of species, in aged mice as compared to the compared group.

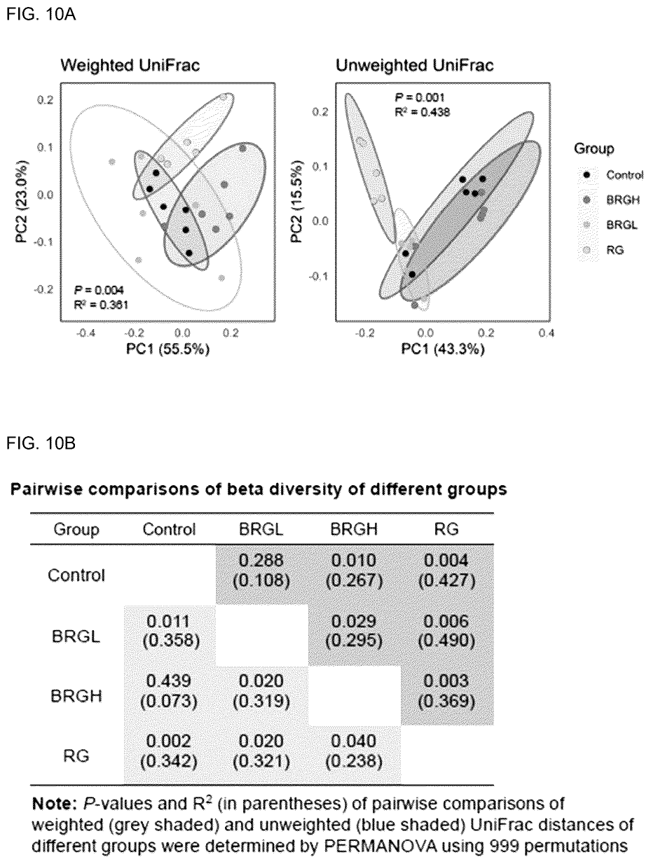

Furthermore, referring to FIG. 10, it can be seen that the administration of BRG at high concentration (BRGH) significantly increased species diversity (beta diversity) in aged mice as compared to the control group.

### Test Example 5: Non-Alcoholic Fatty Liver

To evaluate the long-term effect of BRG intake, 19-month-old aged mice were divided into a normal diet group (NCD; N) and an obesity-induced high-fat/high-calorie diet group (HFHSD), and then BRG was administered ad libitum at different concentrations (150 mg/kg, 300 mg/kg) for 14 weeks. The high-fat/high-calorie diet group was fed with a 49% high-fat diet (hereafter HFD, 49% fat per kcal, Dyets Inc., Bethlethem, PA, USA).

### Four Test Groups

$$Normal diet group ( NCD ) ( n = 10 )$$

$$High ‐ fat / high ‐ calorite diet group ( HFHSD ) ( n = 10 )$$

$$High ‐ fat / high ‐ calorite diet + BRG low ‐ concentration group ( HFHSD + BRGL ) : 150 mg / kg BRG was administered ( n = 10 )$$

$$High ‐ fat / high ‐ calorite diet + BRG high ‐ concentration group ( HFHSD + BRGH ) : 300 mg / kg BRG was administered ( n = 10 )$$

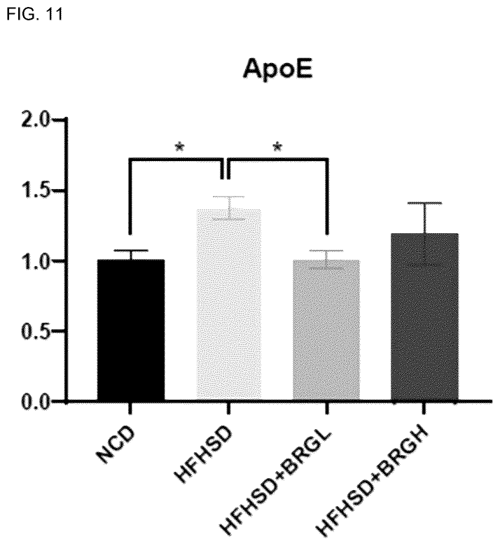

After completion of the 14 weeks of administration, the mice were sacrificed and the expression of ApoE (apolipoprotein E) mRNA in liver tissue was measured (FIG. 11).

Referring to FIG. 11, it can be seen that the expression of the ApoE (apolipoprotein E) gene in liver tissue was significantly increased in the high-fat/high-calorie diet-induced obesity group (HFHSD) as compared to the normal diet group (NCD), while the expression of the ApoE gene was decreased to the level of the normal diet (NCD) for the BRG low-concentration administration group (HFHSD+BRGL), as compared to the high-fat/high-calorie diet obesity induced group (HFHSD).

Furthermore, the liver tissue was fixed in 10% formalin solution, washed with water and dehydrated to make paraffin blocks, which were sectioned and stained with H&E. The amount (area) of lipid droplets accumulated in the liver tissue were observed under an optical microscope at 100× magnification, photographed with an Olympus DP-72 microscopic camera (Olympus, Tokyo, Japan), and quantified. The result is shown in Table 3. The degree of accumulation of lipid droplets in the liver tissue was analyzed by the SCORE evaluation method of a non-alcoholic steatosis model (Kleiner DE et al., 2005).

TABLE 3

| | Lipid droplets | Relative |
|---|---|---|
| | in liver tissue | percentage |
| Score | (score) | (%) |
| Normal diet group (NCD) | 0.0 | — |
| High-fat/high-calorie diet group (HFHSD) | 1.73 | 100 |
| High-fat/high-calorie diet + BRG low- | 0.59** | −65.90** |
| concentration group | | |
| High-fat/high-calorie diet + BRG high- | 0.32*** | −81.50*** |
| concentration group | | |

Referring to Table 3, it can be seen that BRG administration significantly inhibits the production of lipid droplets in liver tissue in obesity-induced aged mice. In particular, the production of lipid droplets in liver tissue was inhibited significantly in the BRG high-concentration group as compared to the BRG low-concentration group.

In addition, blood was collected from the posterior vena cava of the sacrificed animals. The blood was centrifuged at 9,000 rpm for 20 minutes to separate serum. AST and ALT, as indicators of liver inflammation, from the serum sample were analyzed using a biochemical analyzer (Accute TBA-40FR, Toshiba Medical System Co., Japan).

The result is shown in Table 4.

TABLE 4

| Score | AST (IU/L) | ALT (IU/L) |
|---|---|---|
| Normal diet group (NCD) | 73.2 | 26.4 |
| High-fat/high-calorie diet group (HFHSD) | 169.4^{###} | 79.1^{###} |
| High-fat/high-calorie diet + BRG low- | 98.6** | 47.5** |
| concentration group | | |
| High-fat/high-calorie diet + BRG high- | 75.9*** | 28.3*** |
| concentration group | | |

Referring to Table 4, it can be seen that BRG administration (BRG) significantly reduces AST and ALT levels in the obesity-induced aged mice.

These results indicate that the administration of the BRG of the present disclosure is effective in inhibiting fat accumulation in liver tissue and ameliorating liver damage and, therefore, may be useful in the amelioration or treatment of non-alcoholic fatty liver.

Although the present disclosure has been described with the specific exemplary embodiments mentioned above, various modifications or variations may be made without departing from the spirit and scope of the present disclosure. In addition, the modifications or variations are also included in the scope of the appended claims.

What is claimed is:

1. A method for preparing a bioconverted red ginseng extract, comprising a step of enzymatically reacting a red ginseng extract by adding an enzyme mixture of cellulase, β-glucosidase and hemicellulase.

2. The method for preparing a bioconverted red ginseng extract according to claim 1, which further comprises a step of obtaining the red ginseng extract by extracting red ginseng with water, a C₁₋₄ lower alcohol or a mixed solvent thereof.

3. The method for preparing a bioconverted red ginseng extract according to claim 2, wherein the red ginseng is a mixture of red ginseng root and red tail ginseng root at a weight ratio of 80:20 to 40:60.

4. The method for preparing a bioconverted red ginseng extract according to claim 1, wherein the enzyme mixture is derived from Aspergillus niger.

5. The method for preparing a bioconverted red ginseng extract according to claim 4, wherein the enzyme mixture is sumizyme AC.

6. The method for preparing a bioconverted red ginseng extract according to claim 1, wherein the sum of the contents of Rb1, Rg1 and Rg3 is at least 7.0 mg/g, the content of F2 is at least 2.0 mg/g, the content of compound K is at least 1.0 mg/g, the content of ginsenoside Rk1 is at least 5.0 mg/g, and the content of ginsenoside Rg5 is at least 5.0 mg/g.

7. The method for preparing a bioconverted red ginseng extract according to claim 6, wherein the bioconverted red ginseng extract is for use in the preparation of a drug for preventing or treating cholestatic liver disease or non-alcoholic fatty liver.

8. The method for preparing a bioconverted red ginseng extract according to claim 6, wherein the bioconverted red ginseng extract increases the expression level of at least one bile acid biosynthesis-related protein selected from CYP27A1, CYP46A1 and FXRα, or a gene encoding the same.

9. The method for preparing a bioconverted red ginseng extract according to claim 6, wherein the bioconverted red ginseng extract increases the expression level of one or more bile acid transport-related protein selected from CATP1A4, OSTα, SOD2 and SOD3, or a gene encoding the same.

10. The method for preparing a bioconverted red ginseng extract according to claim 6, wherein the bioconverted red ginseng extract suppresses Firmicutes and increases Bacteroidetes among the gut microbiota.

11. The method for preparing a bioconverted red ginseng extract according to claim 6, wherein the bioconverted red ginseng extract increases the relative abundance of at least one strain selected from Parabacteroides goldsteinii and Ruminococcus faecis in the gut.
